(19) United States
(12) Patent Application Publication
(10) Pub. No.: US 2007/0148218 A1
(43) Pub. Date: Jun. 28, 2007

(54) Olanzapine containing transdermal drug delivery compositions

(76) Inventor: Ryan D. Gordon, Edina, MN (US)

Correspondence Address: 3M INNOVATIVE PROPERTIES COMPANY, PO BOX 33427, ST. PAUL, MN, 55133-3427, US

(21) Appl. No.: 10/579,604

(22) PCT Filed: Nov. 2, 2004
(86) PCT No.: PCT/US04/36439

§ 371 Date: May 17, 2006

Related U.S. Application Data
(60) Provisional application No. 60/523,186, filed on Nov. 18, 2003.

(51) Int. Cl. A61K 31/551; A61K 9/70

(52) U.S. Cl. 424/449; 514/220

(57) ABSTRACT

The invention features compositions for the transdermal administration of olanzapine. The compositions include olanzapine or a pharmaceutically acceptable salt thereof, a pressure sensitive adhesive, and an excipient, such as a permeation enhancer and/or a solubilizer of olanzapine. The compositions are useful for the treatment of certain psychiatric disorders, for example schizophrenia and bipolar mania.

### FIELD OF THE INVENTION

The present invention relates to olanzapine containing transdermal drug delivery compositions.

### BACKGROUND OF THE INVENTION

Transdermal administration of drugs is known to have many potential advantages, such as avoidance of first-pass metabolism, avoidance of gastro-intestinal irritation, sustained release, and improved patient compliance with treatment regimens. In treatments of many diseases, including neurological diseases, such as schizophrenia and bipolar disorders, drug non-compliance can be a serious problem, with some reports indicating that as many as two-thirds of patients may be non-adherent or partially adherent to medications.

Olanzapine is known to be useful in the treatment of disorders of the central nervous system. It is commercially available in tablet form under the brand name ZYPREXA® for treatment of schizophrenia and bipolar mania. The chemical designation of olanzapine is 2-methyl-10-(4-methyl-1-piperazinyl)-4H-thieno-[2,3-b][1,5]benzodiazepine. Devices having superabsorbent films and 1,2-butanediol have been used for the transdermal administration of olanzapine, but a need remains for more effective systems for administering olanzapine transdermally.

### SUMMARY OF THE INVENTION

The present invention provides compositions that are suitable for transdermal delivery of olanzapine. In one aspect, the present invention features a transdermal drug delivery composition comprising a pressure sensitive adhesive, an excipient, and olanzapine or a pharmaceutically acceptable salt thereof. The pressure sensitive adhesive includes a copolymer made up of copolymerized monomers, wherein at least one monomer is selected from the group consisting of isooctyl acrylate, ethyl hexyl acrylate, and n-butyl acrylate, and at least one monomer is selected from the group consisting of acrylamide, vinyl acetate, hydroxy ethyl acrylate, and acrylic acid. The excipient is selected from the group consisting of amine oxides, unsaturated fatty acids, isopropyl myristate, lauroglycol, α-terpineol, polyethylene glycol, sorbitan esters, lactic acid and dimethylsulfoxide.

In one embodiment, the compositions of the present invention are substantially free of or free of undissolved olanzapine.

In another embodiment, the compositions of the present invention are adhered to one surface of a backing to create a transdermal drug delivery device.

The transdermal compositions and devices of the invention are useful in the treatment of certain disorders of the central nervous system, including psychiatric disorders such as schizophrenia and bipolar mania. The compositions and/or devices can be applied to the skin of a patient suffering from such a disorder for a period of time sufficient to produce the desire therapeutic result, typically between about 1 and about 7 days. The compositions and devices are able to provide a sustained release delivery of olanzapine without the concerns of patient compliance associated with many other forms of drug delivery.

The above summary is not intended to describe every embodiment or implementation of the present invention. Additional features and advantages of the invention will be apparent from the following detailed description thereof, and from the claims.

### DETAILED DESCRIPTION OF THE INVENTION

The present invention features a transdermal drug delivery composition comprising a pressure sensitive adhesive, an excipient, and olanzapine or a pharmaceutically acceptable salt thereof. The pressure sensitive adhesive includes a copolymer made up of copolymerized monomers, wherein at least one of the monomers is isooctyl acrylate, ethyl hexyl acrylate, or n-butyl acrylate, and at least one of the monomers is acrylamide, vinyl acetate, hydroxy ethyl acrylate, or acrylic acid. The excipient is selected from the group consisting of amine oxides, unsaturated fatty acids, isopropyl myristate, lauroglycol, α-terpineol, polyethylene glycol, sorbitan esters, lactic acid, and dimethylsulfoxide.

The compositions of the present invention further comprise olanzapine or a pharmaceutically acceptable salt thereof. The chemical designation of olanzapine is 2-methyl-10-(4-methyl-1-piperazinyl)-4H-thieno-[2,3-b][1,5]benzodiazepine, and is further described in U.S. Pat. No. 5,229,382 (Chakrabarti et al.), which is incorporated by reference herein in its entirety. Olanzapine may be administered in the form of a pharmaceutically acceptable salt or in the free base form. The free base form is particularly well suited for compositions of the present invention.

The olanzapine may be dissolved or dispersed in the composition, and in one embodiment, the composition is substantially free of or completely free of undissolved olanzapine. The presence of undissolved olanzapine may be detected by examination with a low-power optical microscope (e.g., at 10× to 20× magnification). It should be understood that where only an occasional crystal or undissolved particle is present or where less than about 1% of the total amount of olanzapine is undissolved, the composition is considered to be substantially free of undissolved olanzapine.

The composition typically contains a therapeutically effective amount of olanzapine. This amount will vary according to the form of the drug used, the particular condition to be treated, the amount of time the composition is allowed to remain in contact with the skin of the subject, and other factors known to those of skill in the art. Generally, the amount of drug present in the transdermal drug delivery composition will be about 0.1 to about 40 wt-%, typically about 5.0 to about 25 wt-%, and more typically about 10.0 to about 20.0 wt-% based on the total weight of the composition.

The pressure sensitive adhesives of the present invention are prepared according to well known methods of radical polymerization, described for example in U.S. Pat. No. RE 24,906 (Ulrich), which is incorporated by reference herein in its entirety. The amount of isooctyl acrylate, ethyl hexyl acrylate, and/or n-butyl acrylate monomer in the composition is typically between about 40% and about 98%, more typically between about 60% and about 95%, and most preferably between about 70% and about 90% by weight of the copolymer composition. Isooctyl acrylate and ethyl hexyl acrylate are preferred monomers. Isooctyl acrylate is a particularly preferred monomer. The amount of acrylamide, vinyl acetate, hydroxy ethyl acrylate, and/or acrylic acid monomer in the composition is typically between about 2% and about 60%, more typically between about 5% and about 40%, and most preferably between about 10% and about 30% by weight of the copolymer composition. The copolymers comprising the pressure sensitive adhesive may optionally further comprise other radically polymerizable monomers that are well known in the art. The copolymers comprising the pressure sensitive adhesive may optionally further comprise a substantially linear macromonomer copolymerizable with the other monomers. Suitable macromonomers include polymethylmethacrylate, styrene/acrylonitrile copolymer, polyether, and polystyrene macromonomers.

Suitable excipients for use in the present invention include, but are not limited to, amine oxides, unsaturated fatty acids, isopropyl myristate, lauroglycol, α-terpineol, polyethylene glycol, sorbitan esters, lactic acid, and dimethylsulfoxide. In one embodiment, the excipient is a skin permeation enhancer. Permeation enhancers are desirable excipients for use in transdermal drug delivery, because the skin typically presents an effective barrier to passage of most drug molecules. Amine oxides, unsaturated fatty acids, α-terpineol, polyethylene glycol, and sorbitan monooleate are preferred permeation enhancers. Amine oxides and unsaturated fatty acids are particularly effective permeation enhancers. Amine oxides include, for example, lauramine oxide and 2-hexadecyldimethylamine oxide. Lauramine oxide is a particularly preferred amine oxide. Unsaturated fatty acids include, for example, oleic acid, linoleic acid, and linolenic acid. Oleic acid is a preferred unsaturated fatty acid. Sorbitan esters include, for example, sorbitan monooleate, sorbitan laurate, and sorbitan stearate. Sorbitan monooleate is a preferred sorbitan ester. Isopropyl myristate and lauroglycol are also suitable for use as permeation enhancers. The permeation enhancer should be present in an amount sufficient to allow permeation of a sufficient amount of olanzapine across the skin so as to have a desired therapeutic effect. The amount of permeation enhancer is typically less than about 40% by weight of the total composition and more typically less than about 30%. The permeation enhancers are dispersed, typically substantially uniformly, and more typically dissolved in the composition.

In another embodiment of the invention, the excipient is a solubilizer of olanzapine, i.e., an additive that is capable of dissolving olanzapine or a pharmaceutically acceptable salt thereof. Solubilizers may be used both to increase the amount of total dissolved drug in the composition and/or to increase the solubility of drug in one or more layers of the skin. The solubility of olanzapine in the solubilizer is typically greater than the solubility of olanzapine in the pressure sensitive adhesive. In one embodiment of the invention, the solubilizer is selected from the group consisting of lactic acid and dimethylsulfoxide. The amount of solubilizer used will vary depending on the desired dosing levels and durations, but the amount of solubilizer is typically less than about 35% by weight of the total composition and more typically less than about 25%. The total combined amount of permeation enhancer and solubilizer in the composition is typically less than about 40% by weight of the total composition and more typically less than about 30%. The solubilizers are dispersed, preferably substantially uniformly, and more preferably dissolved in the composition.

Compositions of the present invention may optionally contain other additives or excipients, such as plasticizers, anti-oxidants, crosslinking agents, and colorants. Optional additives are dispersed, preferably substantially uniformly, and more preferably dissolved in the composition

Transdermal drug delivery devices that include compositions of the invention can be made in the form of an article such as a tape, a patch, a sheet, a dressing or any other form known to those skilled in the art. Generally, the device will be in the form of a patch of a size suitable to deliver a selected amount of drug through the skin.

In certain implementations, the device will have a surface area greater than 1 cm², typically greater than 5 cm², and less than 100 cm², typically less than 40 cm².

Devices of the present invention can include a release liner that covers and protects the skin-contacting surface prior to use by a patient. Suitable release liners include, but are not limited to, conventional release liners having a known sheet material, such as a polyester web, a polyethylene web, a polypropylene web, or a polyethylene-coated paper coated with a suitable fluoropolymer or silicone based coating. Devices of the present invention can be packaged individually in a foil-lined pouch for storage, and may alternatively be provided in a rolled or stacked form suitable for use with a dispensing apparatus.

Examples of flexible backing materials employed as conventional tape backings that can be useful for the present invention include those made from polymer films such as polypropylene; polyethylene, particularly low density polyethylene, linear low density polyethylene, metallocene polyethylenes, and high density polyethylene; polyvinyl chloride; polyester (e.g., polyethylene terephthalate); ethylene-vinyl acetate copolymer; polyurethane; cellulose acetate; and ethyl cellulose. Backings that are layered, such as polyethylene terephthalate-aluminum-polyethylene composites, are also suitable. Fabrics and non-wovens are likewise suitable. In one implementation, the backing is a continuous polymeric film that prevents ingress of external moisture into the reservoir layer from activities such as showering and bathing. Examples of such continuous films include, but are not limited to, polyurethane, polyethylene, and polyester.

The backing thickness is typically more than 10 μm, more typically more than 20 μm, and most preferably more than 40 μm. The backing thickness is typically less than 150 μm, more typically less than 125 μm, and most preferably less than 100 μm.

Skin-contacting layer compositions of the invention can be prepared by combining the pressure sensitive adhesive copolymer, drug, permeation enhancer, and optional additives, such as solubilizers, with an organic solvent (e.g., ethyl acetate, isopropanol, methanol, acetone, 2-butanone, ethanol, toluene, alkanes, and mixtures thereof) to provide a coating composition. The mixture is shaken or stirred until a homogeneous coating composition is obtained. The resulting composition is then applied to a release liner using conventional coating methods (e.g., knife coating or extrusion die coating) to provide a predetermined uniform thickness of coating composition. Non-continuous or discontinuous coatings may be prepared using methods such as stripe coating, screen-printing, and ink-jet printing.

In another embodiment, the present invention features a transdermal drug delivery composition that includes olanzapine or a pharmaceutically acceptable salt thereof, in combination with lauramine oxide or oleic acid as a permeation enhancer. Pressure sensitive adhesives, such as those described above, are suitable vehicles for these compositions, but they may alternatively be delivered from other pressure sensitive adhesives commonly used in transdermal drug delivery, such as polyisobutylenes or silicones, or from reservoir-type devices, such as those containing hydroalcoholic gels. These compositions may be prepared and used in transdermal devices as described above.

The device and compositions of the invention can be used to treat psychiatric disorders, such as schizophrenia and bipolar mania. These treatments generally involve providing the transdermal drug delivery compositions described above, and applying the composition to an external part of the human body for a period of time sufficient to achieve the desired therapeutic result. The period of time for such treatment can be between about 6 hours and about 14 days, typically between about 1 day and about 7 days, and more typically between about 1 day and about 4 days.

The following examples are provided to more particularly illustrate various embodiments of the present invention, and are in no way intended to be limiting thereof.

### EXAMPLES

### In Vitro Skin Permeation Test Method

The skin permeation data given in the examples below was obtained using the following test method. The test samples were either transdermal devices having a total area of 2.0 cm². The release liner was removed, and the patch was applied to human cadaver skin and pressed to cause uniform contact with the skin. The resulting patch/skin laminate was placed patch side up across the orifice of the lower portion of a vertical diffusion cell. The diffusion cell was assembled and the lower portion filled with 10 mL of warm (32° C.) receptor fluid (30% w/w/m-pyrol in water) so that the receptor fluid contacted the skin. The sampling port was covered except when in use. In some instances, the test samples were solutions of olanzapine dissolved in an excipient, in which case approximately 2 g of total solution was placed onto a 2.0 cm² piece of skin mounted across the orifice of the lower portion of a vertical diffusion cell.

The cells were maintained at 32±2° C. throughout the course of the experiment. The receptor fluid was stirred by means of a magnetic stirrer throughout the experiment to assure a uniform sample and a reduced diffusion barrier on the dermal side of the skin. The entire volume of receptor fluid was withdrawn at specified time intervals and immediately replaced with fresh fluid. The withdrawn fluid was filtered through a 0.45 μm filter. The last 1-2 mL was then analyzed for olanzapine using conventional high performance liquid chromatography (HPLC). The cumulative amount of olanzapine penetrating through the skin was calculated and reported as μg/cm². Unless noted, the results are reported as the average of 5 replicates.

### Materials

### Preparation of the Copolymers

The copolymers used in the examples that follow were prepared generally according to the methods described below. The inherent viscosity values which are reported below were measured by conventional means using a Cannon-Fenske #50 viscometer in a water bath controlled at 27° C. to measure the flow time of 10 millimeters of the polymer solution (0.15 g of polymer per deciliter of ethyl acetate). The test procedure and apparatus are described in detail in Textbook of Polymer Science, F. W. Billmeyer, Wiley Interscience, Second Edition (1971), pages 84 and 85.

### Preparation of “Dried” Copolymer

Dried copolymer was prepared by knife coating a solution of the copolymer onto a release liner. The coated release liner was oven dried to remove the solvent and reduce the level of residual monomers. The dried copolymer was then stripped off of the release liner and stored in a container until used.

Copolymer A. Preparation of Isooctyl Acrylate/Acrylamide/Vinyl Acetate (75/5/20) Copolymer.

Isooctyl Acrylate/Acrylamide/Vinyl Acetate (75/5/20) copolymer was prepared according to the following procedure. A master batch was prepared by combining isooctyl acrylate (621.0 g), acrylamide (41.4 g), vinyl acetate (165.6 g), 2,2′-azobis(2,4-dimethylpentanenitrile) (1.656 g), ethyl acetate (884.5 g) and methanol (87.48 g). A portion (400 g) of the resulting solution was placed in a 1 quart (0.95 L) amber glass bottle. The bottle was purged for 2 minutes with nitrogen at a flow rate of 1 L per minute. The bottle was sealed and placed in a rotating water bath at 45° C. for 24 hours. The resulting copolymer was diluted with ethyl acetate (183.6 g) and methanol (20.4 g). The percent solids of the resultant copolymer was 30.5%. The inherent viscosity was 1.39 dL/g.

Copolymer B. Preparation of Isooctyl Acrylate/Acrylic Acid (90/10) Copolymer.

Isooctyl Acrylate/Acrylic Acid (90/10) copolymer was prepared according to the following procedure. A flask equipped with an agitator, condenser, nitrogen inlet tube and an addition funnel was charged with isooctyl acrylate (72.0 g), acrylic acid (8.0 g) and ethyl acetate (78.1 g). The mixture was heated to 60° C. with medium agitation and purged with nitrogen to remove oxygen. LUCIDOL® 75 (0.07 g, available from Atofina Chemicals) premixed in ethyl acetate (3.0 g) was added to initiate reaction. The reaction temperature was maintained at 60° C. Ethyl acetate (1.5 g) was added to the polymer solution every 30 minutes until the conversion of isooctyl acrylate to polymer reaches a minimum of 95%, typically 20-30 hours. An additional charge of Lucidol 75 (0.07 g) premixed with ethyl acetate (3.0 g) was added after 5 hours and nine hours reaction time. When 95% minimum reaction conversion was achieved, the resulting polymer solution was diluted with heptane to 20-23% solids, cooled and drained. The inherent viscosity in ethyl acetate at 0.15 g/dl was measured at 1.7-2.0 dl/g.

Copolymer C. Preparation of Isooctyl Acrylate/2-Hydroxyethyl Acrylate/Vinyl Acetate/Elvacite™ 1010 (56/20/18/6) Copolymer Solution.

A solution was prepared by combining vinyl acetate (38.07 g), polymethylmethacrylate macromonomer (12.69 g of ELVACITE™ 1010 available from ICI Acrylics), ethyl acetate (407.95 g) and methanol (21.47 g) in a 1 quart (0.95 L) amber glass bottle and mixing until dissolved. Isooctyl acrylate (118.45 g), 2-hydroxyethyl acrylate (42.3 g), and 2,2′-azobis(2-methylbutyronitrile) (0.3173 g) were then added to this solution. The bottle was purged for 2 minutes with nitrogen at a flow rate of 1 L per minute. The bottle was sealed and placed in a rotating water bath at 57° C. for 24 hours. At 24 hours the bottle was removed from the rotating water bath and unsealed. The inherent viscosity was 1.00 dL/g.

### Example 1

A transdermal drug delivery device was prepared as follows. A mixed solvent stock solution was prepared by mixing acetone (190.2 g), methanol (47.5 g), and trifluoroacetic acid (2.4 g). A solvated olanzapine stock solution was prepared by mixing the mixed solvent stock solution (140.0 g) with olanzapine (5.4100 g). Oleic acid (13.3161 g), isopropyl myristate (6.6597 g), and olanzapine (3.2998 g) were added, and mixed together in a 9.5 dram (40 mL) glass vial to prepare a mixed excipient stock solution.

Copolymer (2.728 g of dried isooctyl acrylate/acrylamide/vinyl acetate (75/5/20) from Copolymer A above), solvated olanzapine stock solution (7.2703 g), and excipient stock solution (1.2857 g) were added and mixed together in a 4 dram (18 μL) glass vial until a uniform coating formulation was obtained. The coating formulation was knife coated at a wet thickness of 14 mil (356 μm) onto a release liner (Daubert 164P silicone coated release liner). The coated liner was oven dried for 20 minutes at 110° F. (43° C.). The resulting coating contained 10.6 percent olanzapine. The coated liner was laminated onto a backing (the non-release coated side of a Daubert 164P liner). The permeation through human cadaver skin was determined using the test method described above. The results are shown in Table 1 below.

### Example 2

A transdermal drug delivery device was prepared as follows. Oleic acid (6.6579 g), isopropyl myristate (13.3533 g), and olanzapine (1.6946 g) were added and mixed together in a 9.5 dram (40 mL) glass vial to prepare a mixed excipient stock solution.

Copolymer (2.740 g of dried isooctyl acrylate/acrylamide/vinyl acetate (75/5/20) from Copolymer A above), solvated olanzapine stock solution (7.2657 g) from Example 1, and excipient stock solution (1.2871 g) were added and mixed together in a 4 dram (18 mL) glass vial until a uniform coating formulation was obtained. The coating formulation was knife coated at a wet thickness of 14 mil (356 μm) onto a release liner (Daubert 164P silicone coated release liner). The coated liner was oven dried for 20 minutes at 110° F. (43° C.). The resulting coating contained 8.6 percent olanzapine. The coated liner was laminated onto a backing (the non-release coated side of a Daubert 164P liner). The permeation through human cadaver skin was determined using the test method described above. The results are shown in Table 1 below.

### Example 3

A transdermal drug delivery device was prepared as follows. Oleic acid (13.340 g), isopropyl myristate (3.363 g), polyethylene glycol 400 (3.337 g), and olanzapine (3.4218 g) were added and mixed together in a 9.5 dram (40 mL) glass vial to prepare a mixed excipient stock solution.

Copolymer (2.7270 g of dried isooctyl acrylate/acrylamide/vinyl acetate (75/5/20) from Copolymer A above), solvated olanzapine stock solution (7.2756 g) from Example 1, and excipient stock solution (1.2872 g) were added and mixed together in a 4 dram (18 mL) glass vial until a uniform coating formulation was obtained. The coating formulation was knife coated at a wet thickness of 14 mil (356 μm) onto a release liner (Daubert 164P silicone coated release liner). The coated liner was oven dried for 20 minutes at 110° F. (43° C.). The resulting coating contained 10.7 percent olanzapine. The coated liner was laminated onto a backing (the non-release coated side of a Daubert 164P liner). The permeation through human cadaver skin was determined using the test method described above. The results are shown in Table 1 below.

### Example 4

A transdermal drug delivery device was prepared as follows. Oleic acid (11.399 g) and lauramine oxide (0.6040 g) were added and mixed together in a 9.5 dram (40 mL) glass vial to prepare a mixed excipient stock solution.

Copolymer (2.732 g of dried isooctyl acrylate/acrylamide/vinyl acetate (75/5/20) from Copolymer A above), solvated olanzapine stock solution (7.2730 g) from Example 1, and excipient stock solution (1.3118 g) were added and mixed together in a 4 dram (18 mL) glass vial until a uniform coating formulation was obtained. The coating formulation was knife coated at a wet thickness of 14 mil (356 μm) onto a release liner (Daubert 164P silicone coated release liner). The coated liner was oven dried for 20 minutes at 110° F. (43° C.). The resulting coating contained 13.7 percent olanzapine. The coated liner was laminated onto a backing (the non-release coated side of a Daubert 164P liner). The permeation through human cadaver skin was determined using the test method described above. The results are shown in Table 1 below.

### Example 5

A transdermal drug delivery device was prepared as follows. A mixed solvent stock solution was prepared by mixing acetone (292.7 g), methanol (73.5 g), and trifluoroacetic acid (3.7 g). A solvated olanzapine stock solution was prepared by mixing the mixed solvent stock solution (69.943 g) with olanzapine (2,6970 g). An excipient stock solution was prepared by mixing oleic acid (22.9709 g) and olanzapine (7.0182 g).

Copolymer (3.815 g of dried isooctyl acrylate/acrylamide/vinyl acetate (75/5/20) from Copolymer A above), solvated olanzapine stock solution (10.1650 g), and excipient stock solution (2.7166 g) were added and mixed together in a 6 dram (27 mL) glass vial until a uniform coating formulation was obtained. The coating formulation was knife coated at a wet thickness of 14 mil (356 μm) onto a release liner (Daubert 164P silicone coated release liner). The coated liner was oven dried for 20 minutes at 110° F. (43° C.). The resulting coating contained 14.4 percent olanzapine. The coated liner was laminated onto a backing (the non-release coated side of a Daubert 164P liner). The permeation through human cadaver skin was determined using the test method described above. The results are shown in Table 1 below.

### Example 6

A transdermal drug delivery device was prepared as follows. A mixed solvent stock solution was prepared by mixing acetone (292.7 g), methanol (73.5 g), and trifluoroacetic acid (3.7 g). Oleic acid (2.6785 g), isopropyl myristate (2.6849 g), dimethylsulfoxide (2.6844 g), and olanzapine (1.6291 g) were added and mixed together in a 9.5 dram (40 mL) glass vial to prepare a mixed excipient stock solution.

Copolymer (2.284 g of dried isooctyl acrylate/acrylamide/vinyl acetate (75/5/20) from Copolymer A above), mixed solvent stock solution (5.4007 g), excipient stock solution (0.8078 g), olanzapine (0.2261 g), and dimethylsulfoxide (0.4627 g) were added and mixed together in a 4 dram (18 mL) glass vial until a uniform coating formulation was obtained. The coating formulation was knife coated at a wet thickness of 14 mil (356 μm) onto a release liner (Daubert 164P silicone coated release liner). The coated liner was oven dried for 10 minutes at 110° F. (43° C.). The resulting coating contained 10.6 percent olanzapine. The coated liner was laminated onto a backing (the non-release coated side of a Daubert 164P liner). The permeation through human cadaver skin was determined using the test method described above. The results are shown in Table 1 below.

TABLE 1
Human Cadaver Skin Permeation

| | Average Cumulative Amount Penetrated (μg/cm²) | | | | | | | | |
|---|---|---|---|---|---|---|---|---|---|
| Example | | | 24 | | | | 120 | 144 | 168 |
| Number | 4 hr | 8 hr | hr | 48 hr | 72 hr | 96 hr | hr | hr | hr |
| 1 | 0.0 | na | 11 | 60 | 135 | 218 | 296 | 365 | 427 |
| 2 | 0.5 | na | 28 | 100 | 176 | 243 | 297 | 344 | 383 |
| 3 | 0.9 | na | 14 | 55 | 113 | 182 | 249 | 316 | 380 |
| 4 | 0.0 | na | 2.7 | 24 | 73 | 145 | 224 | 309 | 392 |
| 5 | na | 0.6 | 7.1 | 51 | 134 | 256 | 382 | 550 | 701 |
| 6 | na | 1.6 | 13 | 59 | 113 | 168 | 218 | 265 | 306 |
| 16 | na | na | 8.5 | 23 | 38 | 52 | na | na | 91 |
| 17 | na | na | 18 | 38 | 62 | 91 | na | na | 185 |

### Example 7

A saturated solution of olanzapine in lactic acid was prepared by adding an excess of olanzapine to lactic acid, mixing the solution for at least 24 hours, and filtering the solution through a 0.45 μm filter to remove any undissolved olanzapine. The olanzapine concentration in the resulting solution was analyzed using conventional HPLC and was approximately 31.2% by weight of the total solution. The permeation through human cadaver skin was determined using the test method described above. The cumulative amount of olanzapine permeation through the skin after 72 hours was 0.4 μg/cm². The average flux rate between 48 and 72 hours was 0.02 μg/cm²/hr.

### Example 8

A saturated solution of olanzapine in oleic acid was prepared by adding an excess of olanzapine to oleic acid, mixing the solution for at least 24 hours, and filtering the solution through a 0.45 μm filter to remove any undissolved olanzapine. The olanzapine concentration in the resulting solution was analyzed using conventional HPLC and was approximately 24.4% by weight of the total solution. The permeation through human cadaver skin was determined using the test method described above. The cumulative amount of olanzapine permeation through the skin after 72 hours was 770.0 μg/cm². The average flux rate between 48 and 72 hours was 15.8 μg/cm²/hr.

### Example 9

A saturated solution of olanzapine in dimethylsulfoxide was prepared by adding an excess of olanzapine to dimethylsulfoxide, mixing the solution for at least 24 hours, and filtering the solution through a 0.45 μm filter to remove any undissolved olanzapine. The olanzapine concentration in the resulting solution was analyzed using conventional HPLC and was approximately 23.9% by weight of the total solution. The permeation through human cadaver skin was determined using the test method described above. The cumulative amount of olanzapine permeation through the skin after 72 hours was 265.9 μg/cm². The average flux rate between 48 and 72 hours was 2.7 μg/cm²/hr.

### Example 10

A saturated solution of olanzapine in polyethylene glycol 400 was prepared by adding an excess of olanzapine to polyethylene glycol 400, mixing the solution for at least 24 hours, and filtering the solution through a 0.45 μM filter to remove any undissolved olanzapine. The olanzapine concentration in the resulting solution was analyzed using conventional HPLC and was approximately 4.5% by weight of the total solution. The permeation through human cadaver skin was determined using the test method described above. The cumulative amount of olanzapine permeation through the skin after 72 hours was 342.9 μg/cm². The average flux rate between 48 and 72 hours was 9.6 μg/cm²/hr.

### Example 11

A saturated solution of olanzapine in Span 80™ was prepared by adding an excess of olanzapine to Span 80, mixing the solution for at least 24 hours, and filtering the solution through a 0.45 μm filter to remove any undissolved olanzapine. The olanzapine concentration in the resulting solution was analyzed using conventional HPLC and was approximately 3.4% by weight of the total solution. The permeation through human cadaver skin was determined using the test method described above. The cumulative amount of olanzapine permeation through the skin after 72 hours was 323.6 μg/cm². The average flux rate between 48 and 72 hours was 6.8 μg/cm²/hr.

### Example 12

A saturated solution of olanzapine in α-terpineol was prepared by adding an excess of olanzapine to α-terpineol, mixing the solution for at least 24 hours, and filtering the solution through a 0.45 μm filter to remove any undissolved olanzapine. The olanzapine concentration in the resulting solution was analyzed using conventional HPLC and was approximately 1.8% by weight of the total solution. The permeation through human cadaver skin was determined using the test method described above. The cumulative amount of olanzapine permeation through the skin after 72 hours was 514.6 μg/cm². The average flux rate between 48 and 72 hours was 11.5 μg/cm²/hr.

### Example 13

A saturated solution of olanzapine in isopropyl myristate was prepared by adding an excess of olanzapine to isopropyl myristate, mixing the solution for at least 24 hours, and filtering the solution through a 0.45 μM filter to remove any undissolved olanzapine. The olanzapine concentration in the resulting solution was analyzed using conventional HPLC and was approximately 0.6% by weight of the total solution. The permeation through human cadaver skin was determined using the test method described above. The cumulative amount of olanzapine permeation through the skin after 72 hours was 663.6 μg/cm². The average flux rate between 48 and 72 hours was 11.8 μg/cm²/hr.

### Example 14

A saturated solution of olanzapine in lauroglycol was prepared by adding an excess of olanzapine to lauroglycol, mixing the solution for at least 24 hours, and filtering the solution through a 0.45 μm filter to remove any undissolved olanzapine. The olanzapine concentration in the resulting solution was analyzed using conventional HPLC and was approximately 2.5% by weight of the total solution. The permeation through human cadaver skin was determined using the test method described above. The cumulative amount of olanzapine permeation through the skin after 72 hours was 760.0 μg/cm². The average flux rate between 48 and 72 hours was 13.1 μg/cm²/hr.

### Example 15

A saturated solution of olanzapine in lauramine oxide and water was prepared by adding an excess of olanzapine to a 30% solution of lauramine oxide in water, mixing the solution for at least 24 hours, and filtering the solution through a 0.45 μm filter to remove any undissolved olanzapine. The olanzapine concentration in the resulting solution was analyzed using conventional HPLC and was approximately 0.7% by weight of the total solution. The permeation through human cadaver skin was determined using the test method described above. The cumulative amount of olanzapine permeation through the skin after 72 hours was 1474.7 μg/cm². The average flux rate between 48 and 72 hours was 22.3 μg/cm²/hr.

### Example 16

A transdermal drug delivery device was prepared as follows. Copolymer (2.562 g of dried isooctyl acrylate/acrylic acid (90/10) from Copolymer B above), acetone (9.9506 g), methanol (2.6912 g), olanzapine (0.4508 g), and tetrahydrofuran (7.6501 g) were added and mixed together in a 6 dram (27 mL) glass vial until a uniform coating formulation was obtained. The coating formulation was knife coated at a wet thickness of 20 mil (508 μM) onto a release liner (Daubert 164P silicone coated release liner). The coated liner was oven dried for 20 minutes at 110° F. (43° C.). The resulting coating contained 15.0 percent olanzapine. The coated liner was laminated onto a backing (the non-release coated side of a Daubert 164P liner). The permeation through human cadaver skin was determined using the test method described above. The results are shown in Table 1 above.

### Example 17

A transdermal drug delivery device was prepared as follows. A mixed solvent stock solution was prepared by mixing acetone (158.443 g), methanol (39.610 g), and trifluoroacetic acid (2.065 g).

Copolymer (2.255 g of dried isooctyl acrylate/2-hydroxyethyl acrylate/vinyl acetate/Elvacite™ 1010 (56/20/18/6) from Copolymer C above), mixed solvent stock solution (16.9549 g), and olanzapine (0.7486 g) were added and mixed together in a 9.5 dram (40 mL) glass vial until a uniform coating formulation was obtained. The coating formulation was knife coated at a wet thickness of 14 mil (356 μm) onto a release liner (Daubert 164P silicone coated release liner). The coated liner was oven dried for 20 minutes at 110° F. (43° C.). The resulting coating contained 24.9 percent olanzapine. The coated liner was laminated onto a backing (the non-release coated side of a Daubert 164P liner). The permeation through human cadaver skin was determined using the test method described above. The results are shown in Table 1 above.

The present invention has been described with reference to several embodiments thereof. The foregoing description of specific embodiments and examples has been provided to illustrate the invention, and is not intended to be limiting of the scope of the invention. It will be apparent to those skilled in the art that many changes can be made to the described embodiments without departing from the spirit and scope of the invention.

All patents, applications, and publications mentioned above are incorporated by reference herein.

We claim:

1. A transdermal drug delivery composition comprising:
(a) a pressure sensitive adhesive comprising a copolymer comprising copolymerized monomers, wherein said monomers comprise a first monomer selected from isooctyl acrylate, ethyl hexyl acrylate, n-butyl acrylate and combinations thereof, and a second monomer selected from acrylamide, vinyl acetate, hydroxy ethyl acrylate, acrylic acid, and combinations thereof;
(b) at least one excipient selected from amine oxides, unsaturated fatty acids, isopropyl myristate, lauroglycol, α-terpineol, polyethylene glycol, sorbitan esters, lactic acid, dimethylsulfoxide, and combinations thereof; and
(c) olanzapine or a pharmaceutically acceptable salt thereof.

2. The transdermal drug delivery composition according to claim 1, wherein the composition is substantially free of undissolved olanzapine.

3. The transdermal drug delivery composition according to claim 1, wherein the second monomer is vinyl acetate.

4. The transdermal drug delivery composition according to claim 1, wherein the olanzapine comprises the free base form.

5. The transdermal drug delivery composition according to claim 1, wherein the excipient is a skin permeation enhancer.

6. The transdermal drug delivery composition according to claim 5, wherein the permeation enhancer is selected from amine oxides, unsaturated fatty acids, α-terpineol, polyethylene glycol, sorbitan esters, and combinations thereof.

7. The transdermal drug delivery composition according to claim 5, wherein the permeation enhancer is an amine oxide or an unsaturated fatty acid.

8. The transdermal drug delivery composition according to claim 7, wherein the amine oxide is lauramine oxide.

9. The transdermal drug delivery composition according to claim 5, wherein the permeation enhancer is an unsaturated fatty acid.

10. The transdermal drug delivery composition according to claim 5, wherein the unsaturated fatty acid is oleic acid.

11. The transdermal drug delivery composition according to claim 1, wherein the excipient is a solubilizer for olanzapine.

12. The transdermal drug delivery composition according to claim 11, wherein the solubilizer is lactic acid.

13. The transdermal drug delivery composition according to claim 11, wherein the solubilizer is dimethylsulfoxide.

14. A device for the transdermal delivery of olanzapine comprising a backing and a composition according to claim 1, said composition being adhered to one surface of the backing.

15. A transdermal drug delivery composition comprising olanzapine or a pharmaceutically acceptable salt thereof, and a permeation enhancer selected from the group consisting of lauramine oxide, oleic acid, and combinations thereof.

16. The transdermal drug delivery composition according to claim 15, further comprising a pressure sensitive adhesive.

17. A method of treatment of schizophrenia or bipolar mania comprising:
(a) providing a transdermal drug delivery composition according to claim 1; and
(b) applying the composition to an external part of the human body for a period of time sufficient to achieve a desired therapeutic result.

18. The method of claim 17, wherein the period of time is between about 1 day and about 7 days.

19. A method of treatment of schizophrenia or bipolar mania comprising:
(a) providing a transdermal drug delivery composition according to claim 16; and
(b) applying the composition to an external part of the human body for a period of time sufficient to achieve a desired therapeutic result.

20. The method of claim 19, wherein the period of time is between about 1 day and about 7 days.
